(19) United States
(12) Patent Application Publication
(10) Pub. No.: US 2026/0224099 A1
(43) Pub. Date: Aug. 6, 2026

(54) AUTOMATED IRRIGATION AND ASPIRATION CONTROL SYSTEM

(71) Applicant: BOSTON SCIENTIFIC SCIMED, INC., Maple Grove, MN (US)

(72) Inventors: Kian S. Lim, Shrewsbury, MA (US); Longquan Chen, Lexington, MA (US); Niraj Prasad Rauniyar, Plymouth, MN (US)

(73) Assignee: BOSTON SCIENTIFIC SCIMED, INC., Maple Grove, MN (US)

(21) Appl. No.: 19/465,639

(22) Filed: Jan. 30, 2026

Related U.S. Application Data
(60) Provisional application No. 63/753,583, filed on Feb. 4, 2025.

(51) Int. Cl. A61B 1/015; A61B 1/00

(52) U.S. Cl. CPC A61B 1/015; A61B 1/00006; A61B 1/00097; A61B 1/00135

(57) ABSTRACT

An endoscope system may include an endoscope having a pressure sensor at a distal end and an access sheath defining a lumen configured to movably receive the endoscope. A fluid management system may include an inflow pump fluidly coupled to the endoscope and an outflow pump fluidly coupled to the access sheath. A controller may control the pump based on pressure measurements from the pressure sensor and a position of the endoscope's distal end relative to the access sheath's distal end. The controller may receive a maximum allowable pressure value and control the pumps to maintain pressure below the maximum value and adjusts pump operation based on the relative position of the endoscope.

### CROSS REFERENCE TO RELATED APPLICATIONS

The present application claims the benefit of U.S. Provisional Patent Application Ser. No. 63/753,583, filed Feb. 4, 2025, the disclosure of which is incorporated herein by reference.

### TECHNICAL FIELD

The disclosure is directed to a fluid management system. More particularly, the disclosure is directed to methods and systems for flow control in a fluid management system.

### BACKGROUND

Flexible ureteroscopy (fURS), gynecology, and other endoscopic procedures require the circulation of fluid for several reasons. For example, lithotripsy procedures may employ irrigation and aspiration techniques in conjunction with ureteroscopy for kidney stone removal. Fluid management systems regulate the flow of irrigation fluid through the working channel of the ureteroscope while providing aspiration through another lumen. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In an example, an endoscope system may include an endoscope having a pressure sensor at a distal end, an access sheath defining a lumen extending from a proximal end to a distal end thereof and configured to movably receive the endoscope within the lumen, and a fluid management system. The fluid management system may include an inflow pump fluidly coupled to the endoscope, an outflow pump fluidly coupled to the access sheath, and a controller configured to control the inflow pump and outflow pump based on pressure measurements from the pressure sensor and a position of the distal end of the endoscope relative to the distal end of the access sheath.

Alternatively or additionally to any of the examples above, in another example, the access sheath may include markings at fixed intervals along its length and the endoscope may include a camera oriented towards the markings.

Alternatively or additionally to any of the examples above, in another example, the controller may be configured to identify a position of the distal end of the endoscope based on a count of the markings.

Alternatively or additionally to any of the examples above, in another example, the system may further include a linear encoder positioned adjacent to the proximal end of the access sheath.

Alternatively or additionally to any of the examples above, in another example, the endoscope may include markings at fixed intervals along its length.

Alternatively or additionally to any of the examples above, in another example, the controller may be configured to maintain different pressure thresholds based on the position of the distal end of the endoscope.

Alternatively or additionally to any of the examples above, in another example, the controller may be configured to maintain a first pressure threshold when the distal end of the endoscope extends distally beyond the distal end of the access sheath and a second pressure threshold when the distal end of the endoscope is proximal to the distal end of the access sheath.

Alternatively or additionally to any of the examples above, in another example, the second pressure threshold may be less than the first pressure threshold.

Alternatively or additionally to any of the examples above, in another example, the controller may be configured to calculate the second pressure threshold based at least in part on a fluid velocity within the access sheath.

Alternatively or additionally to any of the examples above, in another example, the controller may be configured to maintain a third pressure threshold during withdrawal of large stone fragments, wherein the third pressure threshold may be between the first and second pressure thresholds.

Alternatively or additionally to any of the examples above, in another example, the controller may be configured to receive a maximum allowable pressure value.

Alternatively or additionally to any of the examples above, in another example, the controller may be configured to issue an alert when a withdrawal speed of the endoscope exceeds a threshold value.

Alternatively or additionally to any of the examples above, in another example, the controller may be configured to detect a direction of motion and speed of the endoscope relative to the access sheath.

In an example, a method of controlling fluid flow during a medical procedure may include receiving a maximum allowable pressure value, measuring an initial pressure via a pressure sensor at a distal end of an endoscope, controlling an inflow pump and an outflow pump to maintain a measured pressure at or below the maximum allowable pressure value, determining a position of the distal end of the endoscope relative to a distal end of an access sheath, and adjusting operation of the inflow pump and the outflow pump based on the determined position.

Alternatively or additionally to any of the examples above, in another example, the method may include maintaining the measured pressure at a first threshold when the distal end of the endoscope extends beyond the distal end of the access sheath and maintaining the measured pressure at a second threshold when the distal end of the endoscope is within the access sheath.

In an example, an endoscope system may include an endoscope having a pressure sensor at a distal end, an access sheath defining a lumen extending from a proximal end to a distal end thereof and configured to movably receive the endoscope within the lumen, and a fluid management system including an inflow pump fluidly coupled to the endoscope, an outflow pump fluidly coupled to the access sheath, and a controller configured to control the inflow pump and outflow pump based on pressure measurements from the pressure sensor and a position of the distal end of the endoscope relative to the distal end of the access sheath.

Alternatively or Additionally to any of the Examples Above, in Another Example, the Access Sheath May Include Markings at Fixed Intervals Along its Length.

Alternatively or additionally to any of the examples above, in another example, the endoscope may include a camera configured oriented towards the markings.

Alternatively or additionally to any of the examples above, in another example, the controller may be configured to identify a position of the distal end of the endoscope based on a count of the markings.

Alternatively or additionally to any of the examples above, in another example, the system may further include a linear encoder positioned adjacent to the proximal end of the access sheath.

Alternatively or additionally to any of the examples above, in another example, the endoscope may include markings at fixed intervals along its length.

Alternatively or additionally to any of the examples above, in another example, the controller may be configured to maintain different pressure thresholds based on the position of the distal end of the endoscope.

Alternatively or additionally to any of the examples above, in another example, the controller may be configured to maintain a first pressure threshold when the distal end of the endoscope extends distally beyond a distal end of the access sheath and a second pressure threshold when the distal end of the endoscope is proximal to the distal end of the access sheath.

Alternatively or additionally to any of the examples above, in another example, the second pressure threshold may be less than the first pressure threshold.

Alternatively or additionally to any of the examples above, in another example, the controller may be configured to calculate the second pressure threshold based at least in part on a fluid velocity within the access sheath.

Alternatively or additionally to any of the examples above, in another example, the controller may be configured to maintain a third pressure threshold during withdrawal of large stone fragments, wherein the third pressure threshold may be between the first and second pressure thresholds.

In an example, an endoscope system may include an endoscope having a pressure sensor at a distal end, an access sheath defining a lumen extending from a proximal end to a distal end thereof and configured to movably receive the endoscope within the lumen, and a fluid management system comprising a controller, an inflow pump configured to be fluidly coupled to the endoscope, and an outflow pump fluidly coupled to the access sheath. The controller may be configured to receive a maximum allowable pressure value, measure an initial pressure value via the pressure sensor, control the inflow pump and the outflow pump to maintain a measured pressure below the maximum allowable pressure value, and adjust operation of the inflow pump and the outflow pump based on a position of the distal end of the endoscope relative to a distal end of the access sheath.

Alternatively or additionally to any of the examples above, in another example, the controller may be configured to maintain the measured pressure at a first threshold when the distal end of the endoscope extends distally beyond the distal end of the access sheath and at a second threshold when the distal end of the endoscope is within the access sheath.

Alternatively or additionally to any of the examples above, in another example, the second threshold may be less than the first threshold.

Alternatively or additionally to any of the examples above, in another example, the controller may be configured to issue an alert when a withdrawal speed of the endoscope exceeds a threshold value.

In an example, a method of controlling fluid flow during a medical procedure may include receiving a maximum allowable pressure value, measuring an initial pressure via a pressure sensor at a distal end of an endoscope, controlling an inflow pump and an outflow pump to maintain a measured pressure at or below the maximum allowable pressure value, determining a position of the distal end of the endoscope relative to a distal end of an access sheath, and adjusting operation of the inflow pump and the outflow pump based on the determined position.

Alternatively or additionally to any of the examples above, in another example, the method may include maintaining the measured pressure at a first threshold when the distal end of the endoscope extends beyond the distal end of the access sheath and maintaining the measured pressure at a second threshold when the distal end of the endoscope is within the access sheath.

Alternatively or additionally to any of the examples above, in another example, determining the position may include detecting markings on the access sheath with a camera.

Alternatively or additionally to any of the examples above, in another example, determining the position may include using a linear encoder.

Alternatively or additionally to any of the examples above, in another example, the method may include detecting a direction of motion and speed of the endoscope relative to the access sheath.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1A, FIG. 1B, FIG. 1C, FIG. 1D, FIG. 1E, and FIG. 1F illustrate an operating room environment, provisioned for a urological procedure;

FIG. 2 is a schematic view of a portion of a fluid management system and an endoscope of the operating room environment;

FIG. 3 is a schematic cross-sectional view of the illustrative endoscope and access sheath, taken at line 3-3 of FIG. 2;

FIG. 4 is a schematic view of a portion of the fluid management system and the endoscope of the operating room environment having an alternative position tracking system; and

FIGS. 5A and 5B together form a flowchart of an illustrative method for controlling the inflow pump and the outflow pump.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ Elite endoscope device using pressure and/or temperature data from the endoscope or other endoscopic device. The fluid management system may provide fluid to the body. In some cases, the introduction of fluid into the body from the fluid management system may be controlled by limiting the intraluminal pressure (ILP) as measured at a distal end of the endoscope device. Irrigation and aspiration during lithotripsy may help increase the stone-free rate (SFR), reduce thermal tissue injury, and reduce turbidity. SFR is a measure of how many patients are free of stones or stone fragments after a procedure. Recent studies (in both animal and clinical trials) indicate that an access sheath may be used to effectively provide or facilitate irrigation and/or aspiration during lithotripsy. However, using an access sheath for irrigation and/or aspiration may require two people to operate the endoscope system. For example, two or more users may have to control the laser fiber and control the amount of suction while also maneuvering the ureteroscope (advancing or retracting the scope and articulating the distal tip) inside the anatomy. The present disclosure is directed towards systems and methods for controlling irrigation and aspiration through an intelligent fluid management system to improve performance of the fluid management system. While the present disclosure is described with respect to urological procedures, the systems and methods described herein may be used in other anatomies, as desired.

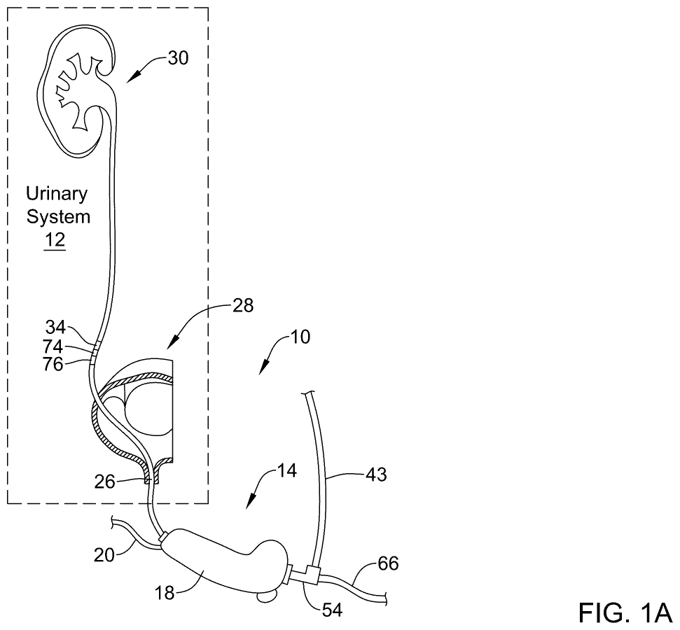

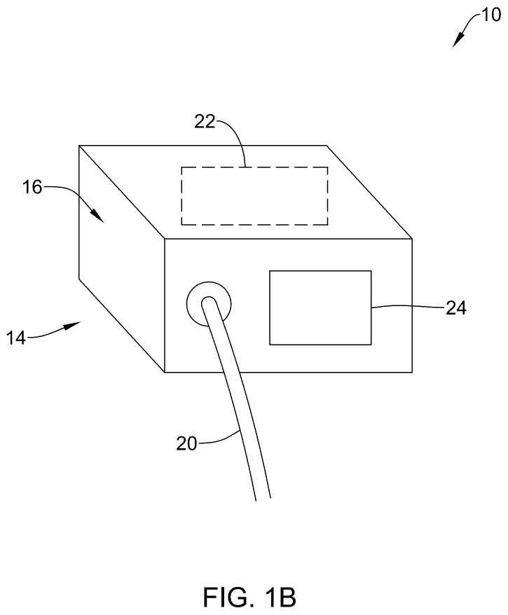

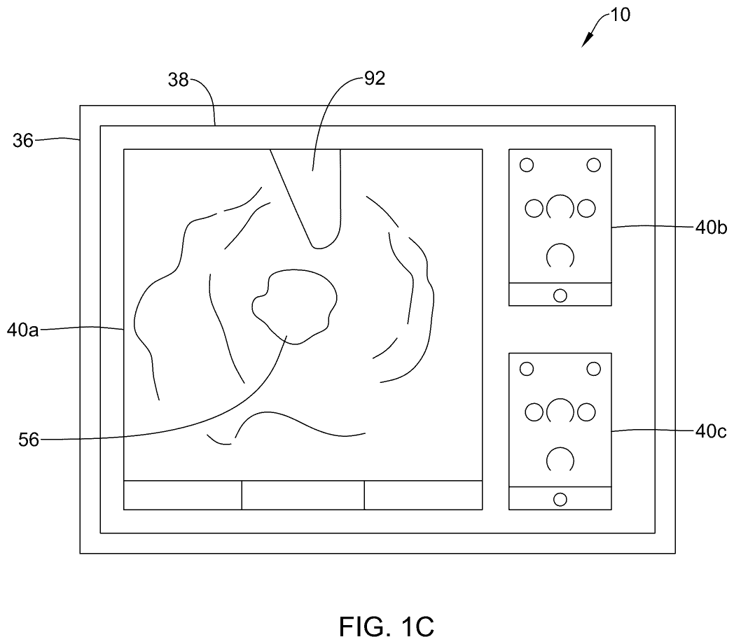

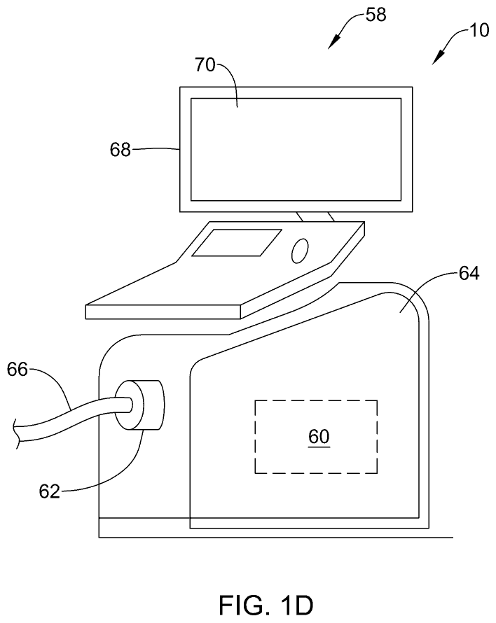

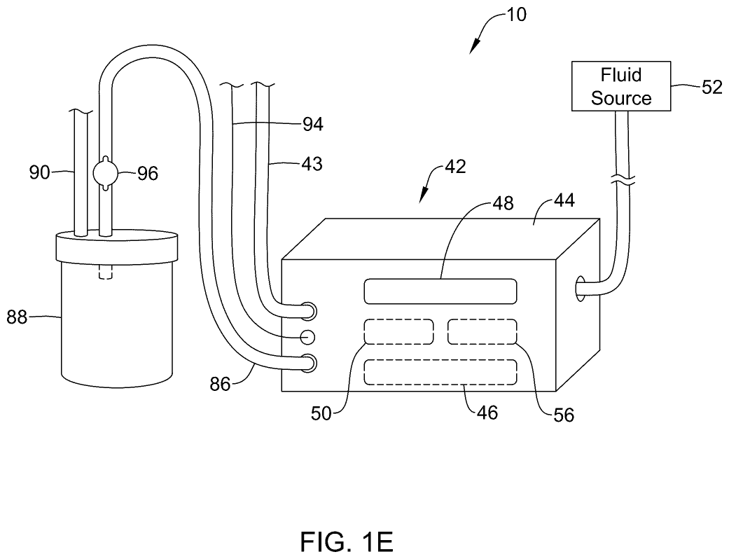

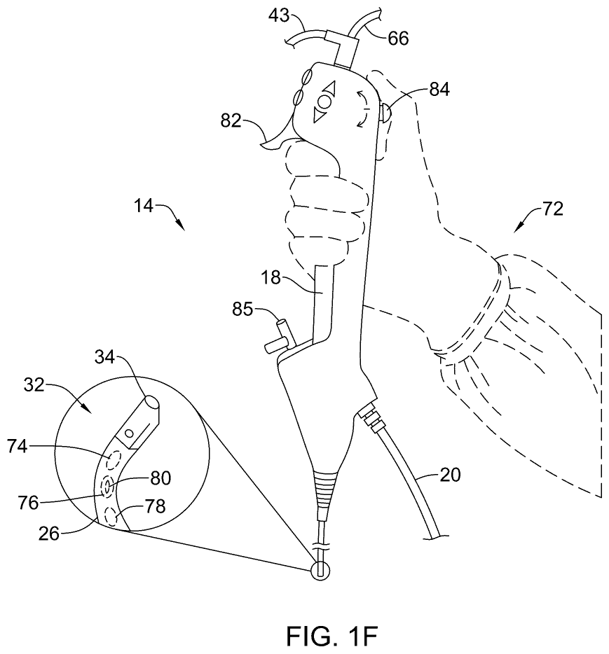

FIGS. 1A-1F illustrate an operating room environment 10, which can be implemented to perform urological procedures. In some cases, the operating room environment may include other devices or features that are not expressly illustrated. The operating room environment 10 is described with respect to a lithotripsy procedure to treat urinary calculi (referred to as “stone”) in a urinary system 12. However, this is not intended to be limiting and the operating room environment 10 could be implemented to perform other urological procedures, such as, for example, percutaneous nephrolithotomy (PCNL), benign prostatic hyperplasia (BPH), transurethral resection of the prostate (TURP), etc.

The operating room environment 10 can be implemented with an endoscope 14, such as, a ureteroscope. The endoscope 14 can include an endoscope console 16, which can be configured to operate with an endoscope handle 18. The endoscope console 16 and endoscope handle 18 can be coupled via connection cable 20. FIG. 1A and FIG. 1F illustrate the endoscope 14 and the endoscope handle 18 in the operating room environment 10 while FIG. 1B illustrates the endoscope console 16. The endoscope 14 can be coupled to a source of power via operating room infrastructure. Further, the connection cable 20 can be configured to provide power from the endoscope console 16 to the endoscope handle 18 and to provide exchange of data between the endoscope console 16 and the endoscope handle 18.

The endoscope console 16 can include a computing system 22 which itself can include (or be coupled to) a display 24 (e.g., touch screen display, or the like). The endoscope console 16 can also include input and/or output devices (not shown), such as buttons, lights, switches, or the like. Further, the operating room environment 10 can include computing components configured to operate as the centralized operating theater controller. An illustrative centralized operating theater controller is described in commonly assigned U.S. Patent Application No. 63/707,003 titled ARCHITECTURE FOR INTEROPERABLE UROLOGY OPERATING ROOM, the disclosure of which is hereby incorporated by reference.

In some embodiments, a single computing system can be provided as part of the endoscope console 16 and configured to operate as both the computing system 22 and a centralized operating theater controller (not explicitly shown). However, this is not intended to be limiting and a centralized operating theater controller could be a separate computing system disposed in the housing of endoscope console 16 or could be integrated into another therapy console provisioned in the operating room environment 10 (e.g., theater display 36, flow management system 42, laser energy console 58, or the like). In other embodiments, a centralized operating theater controller of the operating room environment 10 could be a stand-alone component provisioned in the operating room environment 10. In some embodiments, a centralized operating theater controller can be a cloud computing system (e.g., computing as a service (CaaS), or the like) accessible via a communications network (not explicitly shown). In such an example, equipment in the operating room environment 10 (e.g., computing system 22 of the endoscope console 16, or the like) can include network interfaces to enable communication with a centralized operating theater controller on a communications network.

The endoscope 14 can include an elongate shaft 26 coupled to endoscope handle 18, which can be used to access a patient's bladder 28 and/or kidney 30. In such a procedure, the endoscope 14, and particularly, a distal end 32 of the elongate shaft 26 is inserted into the bladder 28 via the urethra and can be further inserted into the kidney 30 via the ureter, where it can be used to diagnose and/or treat a variety of problems in the urinary system 12. The endoscope 14 can include a camera 34 disposed on the distal end 32 of the elongate shaft 26. The camera 34 can be used to provide a visual feed on a display screen. For example, images captured by the camera 34 can be rendered and displayed on the display 24 of the endoscope console 16. Additionally, the operating room environment 10 can be provided with several other displays (e.g., an internal operating room display and/or an external operating room display) that can be configured to display images and/or video captured by the camera 34 of the endoscope 14.

FIG. 1C illustrates a theater display 36 (or operating theater display), in which is depicted a composite display 38 having a grouping of individual graphical elements 40a, 40b, 40c. For example, the theater display 36 shows a composite display 38 having three graphical elements 40a, 40b, and 40c where a first graphical element 40a depicts a view of an image captured by the camera 34. It is to be appreciated that this view could be a live view or a recorded view.

The operating room environment 10 can further include a fluidics unit or flow management system 42, as shown in FIG. 1E. The flow management system 42 can be coupled to the endoscope 14 and called on to provide fluid flow to and/or from the distal end 32 of the elongate shaft 26. For example, the flow management system 42 may be utilized to clear the visual field of the camera 34. The flow management system 42 can include a console 44. In some examples, the console 44 can be mounted on pole attached to a mobile base (not shown). In other examples, the console 44 can be free standing, table mounted, or the like. The console 44 can include a controller 46 which itself can include a display 48 (e.g., touch screen display, or the like). The flow management system 42 can also include input and/or output devices (not shown), such as, buttons, lights, switches, etc.

The console 44 can include an interface (not shown) with connection sockets and/or busses to which controller 46 can be communicatively coupled to centralized operating theater controller via information technology (IT) infrastructure (not explicitly shown). Such interfaces can also couple the flow management system 42 to a source of power via operating room infrastructure. For example, a connection cable (not shown) could couple the controller 46 to a centralized operating theater controller in the endoscope console 16 and couple the flow management system 42 to power provided by operating room infrastructure.

The flow management system 42 can include a first or inflow pump 50 (disposed in the console 44). The inflow pump 50 can be configured to provide fluid flow to the endoscope 14 when requested by the user (e.g., via the endoscope handle 18, or the like) or by the controller 46. The flow management system 42 can be configured to operate with a cassette (not explicitly shown) and inflow tubing 43. The cassette can be disposed in console 44 via a door. Further, the cassette and inflow tubing 43 can be coupled to a source of fluid 52 and to the endoscope handle 18 (e.g., via a fluid port 54 as shown in FIG. 1A, or the like). In some examples, the fluid source 52 can be saline bags, or the like. Some illustrative fluid cassette and tubing sets are described in commonly assigned U.S. Patent Application Publication No. 2018/0361055, titled AUTOMATED FLUID MANAGEMENT SYSTEM, and U.S. Patent Application No. 63/640,089, titled DEVICES, SYSTEMS, AND METHODS FOR FLOW COMPENSATION IN A FLUID MANAGEMENT SYSTEM, the entire disclosures of which are hereby incorporated by reference. Fluid may flow from the fluid source 52, through the cassette (if so provided), through the inflow fluid tubing 43, and into the endoscope 14 with the flow rate driven by a speed of the inflow pump 50.

The controller 46 can control the inflow pump 50 to cause fluid to flow to the distal end 32 of the elongate shaft 26 via a working channel or dedicated fluid channel (not shown) in the elongate shaft 26. In some examples, the controller 46 may be configured to control the inflow pump 50 to maintain a predetermined pressure at the distal end 32 of the endoscope 14 and/or to maintain a predetermined pressure within the anatomy, as will be described in more detail herein. Additionally, in some embodiments, the flow management system 42 can include a heater and/or a chiller to heat and/or cool the fluid supplied to the treatment site via the elongate shaft 26. Fluid flow to the treatment site (e.g., body cavity, or the like) in the urinary system 12 where the stone is located affects the pressure inside the body cavity. This pressure is referred to herein as intraluminal pressure (ILP).

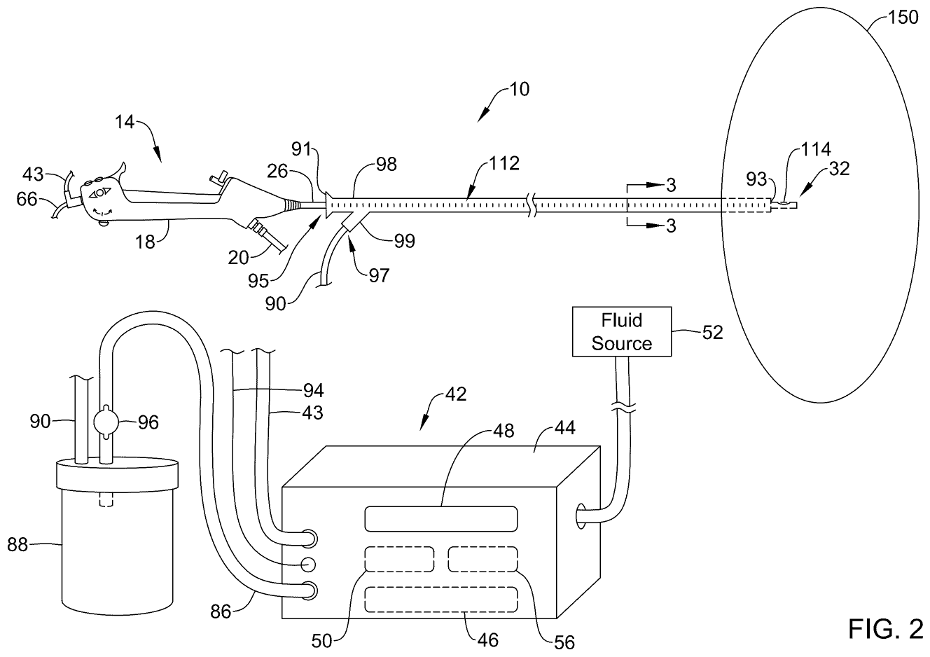

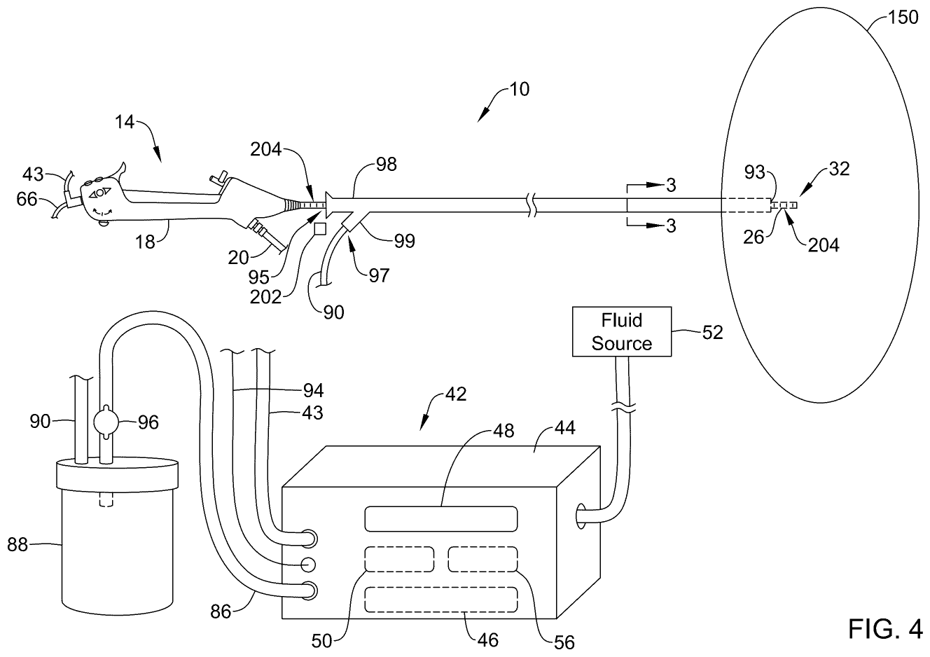

The flow management system 42 can include a second or outflow pump 56 (disposed in the console 44). The outflow pump 56 can be configured to provide suction or aspiration when requested by the user (e.g., via the endoscope handle 18, or the like) or controller 46. The controller 46 can control the outflow pump 56 to apply suction to an access sheath 98 (see, for example, FIG. 2 or 4) or a dedicated channel (not shown) in the elongate shaft 26 to provide suction or remove stone fragments and/or fluid from the anatomy. In some examples, the controller 46 may be configured to control the outflow pump 56 to maintain a predetermined pressure at the distal end 32 of the endoscope 14 and/or to maintain a predetermined pressure within the anatomy, as will be described in more detail herein.

The outflow pump 56 can be in fluid communication with a first suction tube 86 which extends between the fluid management system console 44 and a collection container 88. The collection container 88 may also be fluidly coupled with a second suction tube 90 extending between the collection container 88 and the endoscope 14 or an access sheath (see, for example, FIG. 2 or 4). A speed of the outflow pump 56 may be varied to control the suction generated through the second suction tube 90. An actuatable valve 96 may be provided in line with the first suction tube 86 to selectively fluidly couple the outflow pump 56 with the collection container 88 and the second suction tube 90.

The controller 46 of the fluid management system 42 may be electronically and/or communicatively coupled to the pressure sensor 74 on the distal end 32 of the elongate shaft 26 of the endoscope 14 via a connection 94. In some examples, the connection 94 may extend between the fluid management system console 44 and the endoscope console 16. In other examples, the connection 94 may extend between the endoscope handle 18 and the fluid management system console 44. The connection 94 may be a wired or wireless connection, as desired. The controller 46 of the fluid management system 42 may be configured to control the irrigation fluid and aspiration suction during a procedure, such as lithotripsy procedure, to maintain a desired pressure, as will be described in more detail herein.

During an example lithotripsy procedure, blood and/or debris may be present in the body cavity, which may negatively affect image quality captured by the endoscope 14. Fluid flow (e.g., irrigation fluid flow) from the flow management system 42 may be used to flush the body cavity to improve image the quality. Further, as laser energy (described below) can be used to fragment, ablate, dust, or otherwise treat the stone, heat may be generated at the treatment site. Fluid flow can be used to control the temperature of the treatment site to avoid damage or injury to adjacent tissue.

The operating room environment 10 can further include a laser energy console 58 provisioned in the operating room environment 10, as shown in FIG. 1D. Continuing with the example discussed above where the operating room environment 10 is provisioned for a lithotripsy procedure, the laser energy console 58 could be a medical laser console, such as, a Holmium (Ho) laser or a Thulium (Tm) fiber laser console. As another example, the laser energy console 58 could be a tissue ablation console (e.g., electronic ablation, RF ablation, etc.). With yet another example, the laser energy console 58 could be a laser morcellator. With some embodiments, the operating room environment 10 could be provisioned with multiple laser energy consoles 58 (e.g., a morcellator and stone dusting console, or the like). Further, although not shown, the operating room environment 10 could include other consoles appropriate for the procedure to be performed in the operating room environment 10.

The laser energy console 58 can include a laser generator 60 and an optical coupler 62, both disposed in a housing 64. The laser generator 60 can be configured to generate laser energy appropriate for treating a target tissue (e.g., stone). A treatment fiber 66 can be coupled to the laser generator 60 via the optical coupler 62. In some embodiments, the laser generator 60 can comprise multiple light sources (e.g., a treatment beam, multiple treatment beams, an aiming beam, a diagnostic beam, etc.). Further, the laser generator 60 can often include various optical components and sensors configured to measure characteristics or qualities of the laser energy and its effect on the stone, or adjacent tissue.

The laser energy console 58 can include computing system 68 which itself can include a display 70 (e.g., touch screen display, or the like). Laser energy console 58 can also include input and/or output devices (not shown), such as, buttons, lights, switches, foot pedals, etc.

The housing 64 can include an interface (not shown) with connection sockets and/or busses to which computing system 68 can be communicatively coupled to centralized operating theater controller via IT infrastructure. Such interfaces can also couple laser energy console 58 to a source of power via operating room infrastructure. For example, a connection cable (not shown) could couple computing system 68 to a centralized operating theater controller in the endoscope console 16 (e.g., via IT infrastructure, or the like) and couple the laser energy console 58 to power provided by operating room infrastructure.

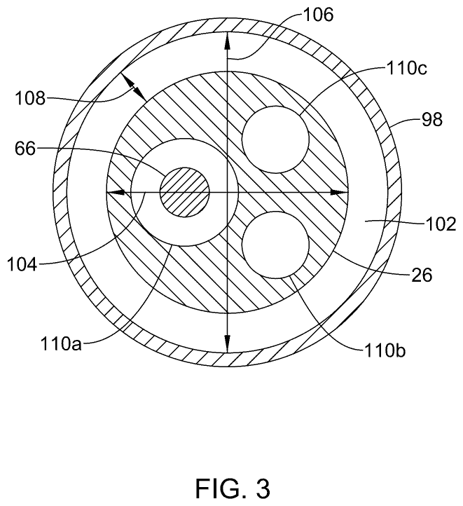

During an example lithotripsy procedure, the treatment fiber 66 can be inserted into port 54 of an endoscope handle 18 and pushed through a working channel 110a (see, for example, FIG. 3) of the elongate shaft 26 such that a distal end 92 (see, for example, FIG. 1C) of the treatment fiber 66 can be positioned proximate to a stone in the urinary system 12. For example, graphical element 40a depicts an image captured by camera 34 of endoscope 14 in which the distal end 92 of the treatment fiber 66 and stone 56 are shown in the urinary system 12. Laser generator 60 can generate laser energy, which is optically coupled to treatment fiber 66 via the optical coupler 62. The laser energy is conveyed through the treatment fiber 66 and emitted from the distal end 92, where it may be incident on stone to cause the stone to be treated (e.g., ablated, fragmented, dusted, or the like).

The computing system 68 can control the laser generator 60 (e.g., responsive to input from endoscope 14, endoscope handle 18, responsive to an input device like a foot pedal, responsive to sensor(s) output, responsive to control signals from centralized operating theater controller, or the like) to cause the laser generator 60 to generate laser energy having parameters appropriate for the treatment to be generated.

In some embodiments, the working channel in which the treatment fiber 66 is inserted is different from the working channel through which fluid supplied by flow management system 42 flows. With some embodiments, the working channel in which the treatment fiber 66 is inserted is the same working channel through which fluid supplied by flow management system 42 flows.

A user (e.g., physician, a nurse, an assistant, or the like) of the operating room environment 10 can configure the operating room environment via the computing components of each respective one of therapy consoles (e.g., the endoscope console 16, the fluid management system 42, the laser energy console 58, or the like) provisioned in the operating room environment 10. For example, a user can configure the endoscope 14 via the endoscope computing system 22, configure the flow management system 42 via the fluid management system controller 46, and configure the laser energy console 58 via the laser energy computing system 68. As another example, a user can configure individual ones of the components of the operating room environment 10 via a centralized operating theater controller.

Further, a user can perform a treatment via one of more of the therapy devices described above. For example, the endoscope 14 includes the endoscope handle 18, which is depicted in use by a user 72 in FIG. 1F. As outlined above, the endoscope handle 18 can be fluidly coupled to the flow management system 42 via a cassette and the inflow fluid tubing 43. Further, a treatment fiber 66 can be disposed through endoscope handle 18 and into urinary system 12. It is to be appreciated that although FIG. 1F depicts a user 72 manipulating endoscope handle 18 during a procedure in the operating room environment 10, other embodiments may provide robotic, non-manual, or non-touch-based control of devices, such as, the endoscope handle 18.

In some embodiments, the endoscope 14 may include one or more sensors, which can be disposed proximate the distal end 32 of the elongate shaft 26. For example, FIG. 1F a depicts pressure sensor 74 at the distal end 32 of the elongate shaft 26. The pressure sensor 74 can be configured to measure an intraluminal pressure (ILP) within the treatment site (see FIG. 1A). The endoscope 14 may also include other sensors such as, for example, a temperature sensor 76, a grating 78 (e.g., a Fiber Bragg grating, or the like) to detect stresses, and/or an antenna or electromagnetic sensor 80 (e.g., a position sensor).

Further, as noted, the endoscope 14 includes at least one camera 34 disposed at the distal end 32 of the elongate shaft 26 to provide a visual feed (e.g., as shown in graphical element 40a, or the like) to the user. The endoscope handle 18 can have a fluid flow on/off switch 82, which allows the user 72 to control when fluid is flowing through the elongate shaft 26 and into the treatment site. The endoscope handle 18 may further include other buttons 84 that perform other functions (e.g., control other devices provisioned in the operating room environment 10, or the like). For example, in some embodiments, the endoscope handle 18 may include buttons 84 to control the temperature of the fluid. In some embodiments, the endoscope handle 18 may also include a drainage port 85, which may be connected to a drainage system (e.g., of operating room infrastructure) and can be configured to provide a path for return flow of fluid from the treatment site.

FIG. 2 is a schematic view of a portion of the fluid management system 42 and the endoscope 14 of the operating room environment 10 in use with a patient 150. The elongate shaft 26 of the endoscope 14 has been inserted into the patient through the lumen of an access sheath 98. The access sheath 98 extends from a proximal end 91 configured to remain outside the patient 150 to a distal end 93 configured to be inserted into the patient 150. A main lumen 102 (see, for example, FIG. 3) through which the endoscope 14 is advanced extends from the proximal end 91 to the distal end 93 of the access sheath 98. The access sheath 98 can include a first distal opening 95 through which the endoscope 14 is inserted. The access sheath 98 may also include a second distal opening 97 positioned at a side port or branch access 99 of the access sheath 98. A lumen of the side port 99 can be in fluid communication with the main lumen 102 of the access sheath 98. The suction tubing 90 may be fluidly coupled with the side port 99 in a fluid-tight manner to fluidly couple a lumen of the suction tubing 90 with the main lumen 102 of the access sheath 98. When the outflow pump 56 of the fluid management system 42 is activated, stones, stone fragments, fluids, or the like may be aspirated into the main lumen 102 of the access sheath 98 and drawn into the collection container 88 via the suction tubing 90.

FIG. 3 is a schematic cross-sectional view of the illustrative endoscope 14 and access sheath 98, taken at line 3-3 of FIG. 2. The elongate shaft 26 of the endoscope 14 has an outer diameter 104 that is less than an inner diameter 106 of the access sheath 98 to define a gap or space 108 between an outer surface of the elongate shaft 26 and inner surface of the access sheath 98 to allow stones, stone fragments, fluids, or the like to be aspirated through the access sheath 98. The elongate shaft 26 of the endoscope 14 may include a plurality of lumens 110a, 110b, 110c extending along a length or portion of a length thereof. In some cases, at least one of the lumens 110a may be a working channel configured to receive additional therapeutic or diagnostic devices therethrough. For example, the treatment fiber 66 of the laser energy console 58 may be advanced through the working channel 110a. The elongate shaft 26 of the endoscope 14 may further include one or more lumens 110b, 110c for providing irrigation fluid, lens wash fluid, gas for insufflation, or the like. In some cases, one or more lumens 110b, 110c may be a light guide or house an imaging device, such as, but not limited to, the camera 34. The endoscope 14 may include more than three or fewer than three lumens 110a-c, as desired. Further, the lumens 110a-c may be arranged as desired.

The fluid management system 42 may be configured to automatically control the flow rate of the irrigation or inflow fluid (e.g., via the inflow pump 50) and the flow rate of the aspiration or outflow fluid (e.g., via the outflow pump 56) without user intervention to allow the aspiration to be provided through the access sheath 98 without requiring a second user. Generally, the fluid management system 42 may be preset to maintain a predetermined threshold pressure inside of the ureter or kidney to prevent over pressurization. The predetermined pressure may be maintained through a combination of the first or inflow pump 50 and the second or outflow pump 56. The fluid management system controller 46 of the fluid management system 42 may be configured to compare the intraluminal pressure obtained or measured at the pressure sensor 74 to the predetermined threshold pressure and control the inflow pump 50 and the outflow pump 56 to maintain the predetermined threshold pressure.

When an access sheath 98 is used during a lithotripsy procedure, suction may be applied in the access sheath 98 while a fluid such as, but not limited to, saline, may be flowed through the endoscope 14. The stone fragments, stones, fluids, or the like may be suctioned through the gap 108 between the access sheath 98 and the endoscope 14. However, too much suction pressure may collapse the access sheath 98 to the endoscope 14 and inhibit the flow of stones, stone fragments, fluids, or the like inside the access sheath 98. Maintaining a predetermined optimal pressure within the access sheath 98 may help prevent the access sheath 98 from collapsing. The pressure may be based at least partially on a maximum allowable intraluminal pressure, an outer diameter 104 of the endoscope 14, and an inner diameter 106 of the access sheath 98.

When the stone fragments are suctioned into the access sheath 98 with the distal tip 32 of the endoscope 14 extending distally beyond the distal end 93 out of the access sheath 98, the speeds of the inflow and/or outflow pumps 50, 56 may be set to maximize the effect of removing the stone fragments. However, when the distal tip 32 of the endoscope 14 is disposed within the lumen 102 of the access sheath 98, the inflow and/or outflow pumps 50, 56 are operated to maintain a lower pressure at the distal end 32 of the endoscope 14 with the same flow rate even when there is no change in the intraluminal pressure inside the anatomy. It is further contemplated that as the endoscope 14 is withdrawn into the lumen 102 of the access sheath 98, large stones or stone fragments (e.g., stones or fragments having a cross-sectional dimension greater than the space 108 between the outer surface of the endoscope 14 and the inner surface of the access sheath 98) may be suctioned into the lumen 102 of the access sheath 98. In this instance, the pressure at the distal end 32 of the endoscope 14 will be lower than the pressure when the endoscope 14 extends distally beyond distal end 93 of the access sheath 98 but greater than the pressure when the distal tip 32 of the endoscope 14 is disposed within the lumen 102 of the access sheath 98. This may be at least partially attributed to a reduced (or no) flow rate of irrigation fluid from the endoscope 14 to replenish fluid in the anatomy.

As the pressure within the access sheath 98 may vary with the position of the endoscope 14, it may be desirable for the fluid management system controller 46 of the fluid management system 42 to know the position of the distal tip 32 of the endoscope 14 relative to the distal end 93 of the access sheath 98 to determine how to control the irrigation and outflow pumps 50, 56 to maintain the appropriate pressure at the distal end 32 of the endoscope 14. In some examples, the endoscope 14 may be configured to detect visual markings 112 on the access sheath 98 as the endoscope 14 is moved proximally and/or distally within the access sheath 98. It is contemplated that markings 112 may be formed on the access sheath 98 at fixed intervals. In some cases, the markings 112 may be provided at 1-millimeter (mm) (0.04 inch) intervals. However, the intervals may be less than 1 mm (0.04 inches) or greater than 1 mm (0.04 inches), as desired. In some embodiments, the markings 112 may be formed on an inner or luminal surface of the access sheath 98. The markings 112 may be visual indicia formed by applying a marker element having a different color from the body portion of the access sheath 98. In another example, the markings 112 may be notches or other structural modifications formed in the body portion of the access sheath 98. In other examples, the markings 112 may be applied or formed on an outer surface of the access sheath 98. In such an example, the access sheath 98 may be formed from a transparent material. The markings 112 may extend around an entire circumference of the inner or outer surface of the access sheath 98. In other configurations, the markings 112 may extend around less than an entire circumference of the inner or outer surface of the access sheath 98. It is further contemplated that two or more markings 112 may be positioned at different circumferential locations at a same axial location. These are just some examples of possible arrangements of the markings 112.

A camera 114 mounted on or within the endoscope 14 may be used to track the markings 112 as the endoscope 14 is distally advanced or proximally retracted within the access sheath 98. The camera 114 is communicatively coupled with the endoscope computing system 22 and/or the fluid management system controller 46. In some configurations, the camera 114 may be mounted on or relative to the endoscope 14 such that the field of view of the camera 114 is directed radially outwards towards the inner surface of the access sheath 98. In some examples, the camera 114 may be positioned adjacent to or near the distal tip 32 of the endoscope 14. However, this is not required. The camera 114 may be provided at other axial locations along a length of the endoscope 14, as desired. In some embodiments, the camera 34 which is disposed on the distal end 32 of the endoscope 14 may be used to track the markers 112. In other examples, a separate tracking camera 114 may be provided. While the markers 112 are shown and described on the access sheath 98, in some configurations, the markers 112 may be positioned on the outer surface of the endoscope 14 and the camera 114 may be positioned at or near the proximal end 91 of the access sheath 98.

The endoscope computing system 22 and/or the fluid management system controller 46 may include a visual recognition module. As the camera 114 passes the markings, the visual recognition module maintains a count of each of the markings 112. The count is increased incrementally by 1 with each marking 112 as the endoscope 14 is moved distally and decreased incrementally by 1 with each marking 112 as the endoscope 14 is moved proximally. When the count is equal to or greater than a predetermined number, the visual recognition module determines the distal end 32 of the endoscope 14 is positioned distally beyond the distal end 93 of the access sheath 98. The predetermined number may be determined based on a length of the access sheath 98, a distance between adjacent markings 112, and a position of the camera 114 relative to the distal end 32 of the endoscope 14. When the count is less than the predetermined number, the visual recognition module determines the distal end 32 of the endoscope 14 is positioned proximal to the distal end 93 of the access sheath 98 (or within the lumen 102). The position of the distal end 32 of the endoscope 14 is transmitted to the fluid management system controller 46 if the processing did not occur there. The position of the distal end 32 of the endoscope 14 may then be used to help determine control parameters for the inflow pump 50 and the outflow pump 56. In addition to determining a position of the distal end 32 of the endoscope 14, the camera 114 may also determine a speed of travel of the endoscope 14 with respect to the access sheath 98 and/or a direction of motion (e.g., distal advancement or proximal retraction) of the endoscope 14.

It is contemplated that other systems or methods may be used to track the position of the distal end 32 of the endoscope 14 relative to the distal end 93 of the access sheath 98. FIG. 4 is a schematic view of a portion of the fluid management system 42 and the endoscope 14 of the operating room environment 10 in use with a patient 150 having an alternative system of tracking a position of the end 32 of the endoscope 14 relative to the distal end 93 of the access sheath 98. The system of FIG. 4 may include a linear encoder 202 positioned at or adjacent to the proximal end 91 of the access sheath 98. The linear encoder 202 may be configured to measure the linear movement of the endoscope 14 by reading a scale or markings 204 formed or positioned on an outer surface of the elongate shaft 26 of the endoscope 14. The linear encoder 202 may be optical, magnetic, inductive, capacitive, or laser.

The linear encoder 202 may output a signal that indicates the position of the distal end 32 of the endoscope 14 relative to the distal end 93 of the access sheath 98. The position of the distal end 32 of the endoscope 14 is transmitted to the fluid management system controller 46 if the processing did not occur there. The position of the distal end 32 of the endoscope 14 may then be used to help determine control parameters for the inflow pump 50 and the outflow pump 56. In addition to determining a position of the distal end 32 of the endoscope 14, the linear encoder 202 may also determine a speed of travel of the endoscope 14 with respect to the access sheath 98 and/or a direction of motion (e.g., distal advancement or proximal retraction) of the endoscope 14.

Generally, the fluid management system controller 46 may be configured to operate the outflow pump 50 and the inflow pump 56 based on a measured pressure, one or more threshold pressures Pₐ, P_{f}, Pₛ (configured to prevent over-pressurization), a position of the distal end 32 of the endoscope 14 with respect to the distal end 93 of the access sheath 98, and whether or not laser lithotripsy is active. The fluid management system controller 46 may be configured to receive parameter such as a maximum allowable intraluminal pressure (Pₐ), an outer diameter 104 of the endoscope 14, and an inner diameter 106 of the access sheath 98 from the user. Once the endoscope 14 has been inserted into the patient 150 (as confirmed by visual detection with a camera 114 (FIG. 2) or by linear encoder 202 (FIG. 4)), the fluid management system controller 46 may register the localized anatomy pressure (Pₒ) as measured at the pressure sensor 74. The fluid management system controller 46 checks if the initial pressure Pₒ is less than Pₐ and if saline is flowing. When conditions are met, the fluid management system controller 46 activates the outflow pump 56 to maintain the measured pressure at (or below) Pₐ. The fluid management system controller 46 may be configured to continually monitor the flow velocity of the inflow (irrigation) fluid, the flow velocity of the outflow (aspiration) fluid, whether or not the endoscope 14 is inside the sheath, whether or not the distal end 32 of the endoscope 14 extends distally beyond the distal end 93 of the access sheath 98, and if laser lithotripsy is active. During withdrawal of the endoscope 14, the fluid management system controller 46 may calculate the desired pressure Pₛ at the distal end 32 of the endoscope. If the measured pressure is less than a second threshold pressure P_{f}, the fluid management system controller 46 is configured to alert the user slow the removal of the endoscope. For example, if the measured pressure is than the second threshold pressure P_{f}, the withdrawal speed of the endoscope 14 may exceed a threshold value. If the pressure is greater than the desired pressure Pₛ, the fluid management system controller 46 is configured to gradually activate the inflow pump 50 and the outflow pump 56. The fluid management system controller 46 may continue monitoring until the endoscope 14 is fully retracted from the access sheath 98. The process ends with stones or stone fragments deposited in the collection container 88. Throughout the process, the fluid management system controller 46 may maintain the measured pressure at the pressure sensor 74 within specific thresholds following the relationship: P_{f}<Pₛ<Pₒ<Pₐ, where P_{f} is the threshold pressure when the distal end 32 of the endoscope 32 is inside the access sheath 98, Pₛ is the desired pressure during stone withdrawal, Pₒ is initial measured intraluminal pressure, and Pₐ is maximum allowable intraluminal pressure. The fluid management system controller 46 may automatically adjust the inflow pump 50 and/or the outflow pump to maintain optimal pressure based on the endoscope's position and operation phase, freeing the doctor from manual pressure control while ensuring safe and effective stone removal.

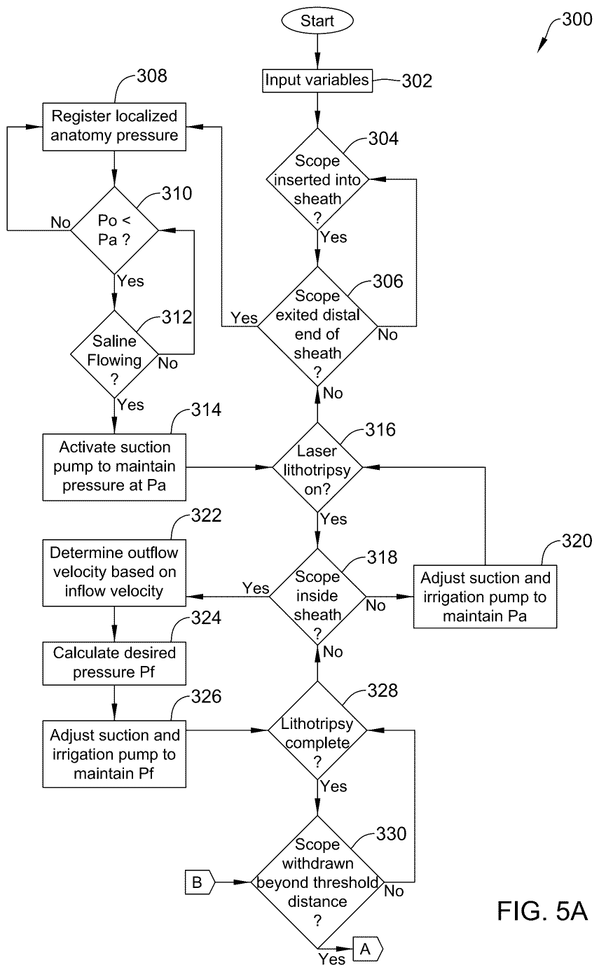

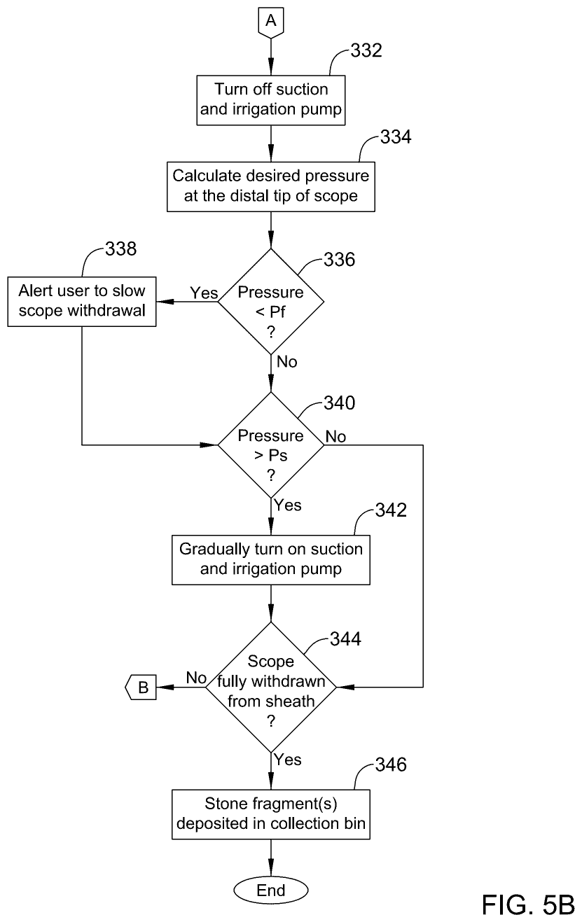

FIGS. 5A and 5B together form a flowchart of an illustrative method 300 for controlling the inflow pump 50 and the outflow pump 56 without requiring the user to manually control the flow of fluid or suction. While certain steps are shown as a sequence within the flowchart, in other embodiments fewer steps are contemplated and the order by which steps are performed can be different than what is illustrated. In some cases, some steps may occur simultaneously or substantially simultaneously. The method 300 will described with the processing occurring within or at the fluid management system controller 46. However, the processing may occur at a central control unit or at other control units within the operating room environment 10. To begin, the user may input variables specific to the operating room environment 10 and the patient into the fluid management system controller 46, as shown at block 302. For example, the user may input the maximum allowable intraluminal pressure (Pₐ), an outer diameter 104 of the endoscope 14, and an inner diameter 106 of the access sheath 98. In some embodiments, the maximum allowable intraluminal pressure Pₐ may be in the range of about 50 millimeters of mercury (mmHg) to about 60 mmHg. However, this is not required. The fluid management system controller 46 may then determine if the endoscope 14 has been inserted into the access sheath 98, as shown at block 304. For example, this may be performed by using the markings 112 and camera 114 system of FIG. 2 or the linear encoder 202 system of FIG. 4 to determine a relative position of the distal end 32 of the endoscope 14. For example, a count of zero may indicate the endoscope 14 has not been inserted into the access sheath 98. Once the fluid management system controller 46 determines that the endoscope 14 is within the lumen 102 of the access sheath 98, the fluid management system controller 46 may determine if the distal end 32 of the endoscope 14 has exited the access sheath 98 (e.g., the distal end 32 of the endoscope 14 extends distally beyond the distal end 93 of the access sheath 98), as shown at block 306. The fluid management system controller 46 may be configured to repeat steps 304 and 306 until the fluid management system controller 46 determines that the distal end 32 of the endoscope 14 has exited the access sheath 98.

Once the distal end 32 of the endoscope 14 has exited the access sheath 98, the fluid management system controller 46 may register the localized or current intraluminal pressure (Pₒ) via the pressure sensor 74 at the distal end 32 of the endoscope 14, as shown at block 308. The fluid management system controller 46 may then compare the measured intraluminal pressure Pₒ to the maximum allowable intraluminal pressure Pₐ, as shown at block 310. If the measured intraluminal pressure Pₒ is not less than the maximum allowable intraluminal pressure Pₐ, the fluid management system controller 46 may continue to repeat steps 308 and 310 until the initial intraluminal pressure Pₒ is less than maximum allowable intraluminal pressure Pₐ. Once the initial intraluminal pressure Pₒ is less than maximum allowable intraluminal pressure Pₐ, the fluid management system controller 46 may determine if irrigation fluid (e.g., water, saline, etc.) is flowing, as shown at block 312. If irrigation fluid is not flowing, the fluid management system controller 46 may be configured to compare the measured intraluminal pressure to the maximum allowable intraluminal pressure Pₐ at predetermined intervals. If at any point, the measured intraluminal pressure is greater than the maximum allowable intraluminal pressure Pₐ, the method may return to step 308.

If irrigation fluid is flowing (e.g., the inflow pump 50 is active), the fluid management system controller 46 may activate the outflow pump 56 to maintain the measured intraluminal pressure less than or equal to the maximum allowable intraluminal pressure Pₐ, as shown at block 314. The fluid management system controller 46 may be configured to increase or decrease the motor speed of the outflow pump 56 to maintain the measured intraluminal pressure Pₒ at a value less than or equal to the maximum allowable intraluminal pressure Pₐ. Next, the fluid management system controller 46 may determine if the treatment fiber 66 is on or active, as shown at block 316. For example, the fluid management system controller 46 may be in communication with the laser generator 60. If the treatment fiber 66 is not active, the method may return to step 306 and repeat the previous steps. If the treatment fiber 66 is active, the fluid management system controller 46 is configured to determine a position of the distal end 32 of the endoscope 14 relative to the distal end 93 of the access sheath 98. For example, the fluid management system controller 46 is configured to determine if the distal end 32 of the endoscope 14 is inside the access sheath 98, as shown at block 318. If the distal end 32 of the endoscope 14 is not within the access sheath 98 (e.g., the distal end 32 of the endoscope 14 extends distally beyond the distal end 93 of the access sheath 98), the fluid management system controller 46 controls the inflow pump 50 and/or the outflow pump 56 to maintain the measured intraluminal pressure Pₒ less than or equal to the maximum allowable intraluminal pressure Pₐ, as shown at block 320. For example, a speed of the inflow pump 50 may be increased and/or a speed of the outflow pump 56 decreased to increase the measured intraluminal pressure. The speed of the inflow pump 50 may be decreased and/or a speed of the outflow pump 56 increased to decrease the measured intraluminal pressure Pₒ. Maintaining flow balance between the irrigation fluid and the aspiration may minimize retropulsion.

The flow balance between the irrigation fluid (flow into the body through the endoscope 14) and the aspiration fluid (flow out of the body through the access sheath 98) may be represented by Equation 1:

$${V}_{1} ( {A}_{1} - {A}_{2} ) = {V}_{2} {A}_{3} \qquad Equation 1$$

where V₁ is the velocity of the aspiration fluid, A₁ is the cross-sectional area of the lumen 102 of the access sheath 98, A₂ is the cross-sectional area of the elongate shaft 26 of the endoscope 14, V₂ is the velocity of the irrigation fluid, and A₃ is the cross-sectional area of the irrigation lumen of the endoscope 14. Said differently, to maintain a substantially constant intraluminal pressure, the volumetric flow rate of fluid exiting the body (aspiration fluid) equals the volumetric flow rate of fluid entering the body (irrigation fluid). As A₁, A₂, A₃, and V₂ are known, the flow velocity V₁ of the aspiration fluid needed to balance fluid flow can be easily calculated. The fluid management system controller 46 may be configured to control a speed of the outflow pump 56 to achieve the desired flow velocity V₁. In some cases, the volumetric flow rate of the irrigation fluid (V₂*A₃) is in the range of about 50 milliliters per minute (mL/min) to about 150 mL/min.

If the distal end 32 of the endoscope 14 is within the access sheath 98 (e.g., the distal end 32 of the endoscope 14 is proximal to the distal end 93 of the access sheath 98), the fluid management system controller 46 may be configured to calculate a new threshold pressure P_{f} to be maintained at the distal end 32 of the endoscope 14 (as measured at the pressure sensor 74). First, the fluid management system controller 46 may calculate or determine the velocity V₁ of the outflow or aspiration fluid based on the velocity V₂ of the inflow or irrigation fluid, as shown at block 322. For example, Equation 1 may be used to determine the velocity V₁ of the outflow or aspiration fluid. Next, the threshold pressure P_{f} to be maintained at the distal end 32 of the endoscope 14 can be calculated or determined, as shown at block 324. The threshold pressure P_{f} for when the distal end 32 of the endoscope 14 is within access sheath 98 may be calculated or determined using Bernoulli's equation.

Bernoulli's equation is a fundamental principle in fluid dynamics that describes the relationship between pressure, velocity, and elevation in a flowing fluid. The equation states that in a steady flow of an incompressible, inviscid fluid, an increase in the velocity of the fluid occurs simultaneously with a decrease in pressure or a decrease in the fluid's potential energy. The equation is mathematically expressed as:

$$P + \frac{1}{2} ρ {V}^{2} + ρ gh = constant \qquad Equation 2$$

where P is the pressure, ρ(rho) is the fluid density, V is the fluid velocity, g is the acceleration due to gravity, and h is the elevation height. Generally, ½ρV² represents kinetic energy per unit volume and μgh represents potential energy per unit volume. While the equation assumes ideal conditions, the equation may be used to approximate the fluid behavior in the endoscope 14 and access sheath 98. When fluid flows through a constricted area (like the gap 108 between the endoscope 14 and access sheath 98), the velocity increases and consequently the pressure decreases. For example, the inflow fluid flow and the outflow fluid flow may be represented as:

$${P}_{f} + \frac{1}{2} ρ {V}_{1}^{2} + ρ gh = {P}_{a} + \frac{1}{2} ρ {V}_{2}^{2} + ρ gh \qquad Equation 3$$

where P_{f} is the threshold pressure when the distal end 32 of the endoscope 14 is within the access sheath 98, V₁ is the velocity of the aspiration fluid, Pₐ is the threshold pressure when the distal end 32 of the endoscope 14 extends distally beyond the distal end 93 of the access sheath 98, and V₂ is the velocity of the irrigation fluid. There may be an insignificant difference in the potential energy for each side of Equation 3. Further, there may be an insignificant volume of fluid reabsorbed within the body of the patient. Generally, when the distal end 32 of the endoscope is inside the access sheath 98, the second threshold pressure P_{f} will be lower with the same flow rate compared to when the distal end is outside the access sheath 98, due to the Bernoulli Effect where increased fluid speed through the restricted area between the scope and sheath results in decreased internal pressure.

Once the fluid management system controller 46 has calculated the new or second threshold pressure P_{f}, the fluid management system controller 46 can control the inflow pump 50 and the outflow pump 56 to maintain the pressure measured at the pressure sensor 74 at or approximately equal to P_{f}, as shown at block 326. As the fluid management system controller 46 is controlling the inflow pump 50 and the outflow pump 56, the fluid management system controller 46 may be configured to determine if the lithotripsy procedure is complete, as shown at block 328. If the lithotripsy procedure is not complete, the fluid management system controller 46 is configured to continually or at predefined intervals determine if the distal end 32 of the endoscope 14 is disposed within the access sheath 98 (block 318). The fluid management system controller 46 may switch between using the maximum threshold pressure Pₐ or the second threshold pressure P_{f} to control the inflow pump 50 and the outflow pump 56 based on the position of the distal end 32 of the endoscope 14 relative to the distal end 93 of the access sheath 98. The second threshold pressure P_{f} may be less than the maximum threshold pressure Pₐ.

If the lithotripsy procedure is complete (e.g., the laser energy console 58 is no longer active), the fluid management system controller 46 may be configured to monitor the position of the distal end 32 of the endoscope 14 to determine when the endoscope 14 has been proximally retracted or withdrawn beyond a threshold distance, as shown at block 330. If the endoscope 14 has not been proximally retracted or withdrawn beyond a threshold distance, the fluid management system controller 46 may be configured to monitor the status of the lithotripsy procedure (block 328) and the withdrawal distance of the endoscope 14 until the endoscope 14 is withdrawn beyond the threshold distance. The threshold distance may be determined by the length of the endoscope 14 and a length of the access sheath 98. Once the endoscope 14 is withdrawn beyond the threshold distance, the fluid management system controller 46 may be configured to turn off or stop the inflow pump 50 and the outflow pump 56, as shown at block 332. At this time the user may be slowly withdrawing the endoscope 14 through the access sheath 98 to pull larger stones or stone fragments (e.g., having a cross-sectional dimension greater than the gap 108 between the endoscope 14 and the access sheath 98) into the access sheath 98. The fluid management system controller 46 may be configured to calculate or determine the desired pressure Pₛ at the distal end 32 of the endoscope 14 during this portion of the procedure, as shown at block 334. Again, Bernoulli's equation can be used to facilitate determination of the desired pressure Pₛ during larger stone or stone fragment removal as shown:

$${P}_{f} + \frac{1}{2} ρ {V}_{1}^{2} + ρ gh = {P}_{s} + \frac{1}{2} ρ {V}_{2}^{2} + ρ gh \qquad Equation 4$$

where P_{f} is the threshold pressure when the distal end 32 of the endoscope 14 is within the access sheath 98, V₁ is the velocity of the aspiration fluid, Pₛ is the desired pressure at the distal end 32 of the endoscope 14 during withdrawal of the endoscope 14, and V₂ is the velocity of the irrigation fluid. There may be an insignificant difference in the potential energy for each side of Equation 4. Further, there may be an insignificant volume of fluid reabsorbed within the body of the patient.

The fluid management system controller 46 may be configured to monitor the pressure measured at the pressure sensor 74 at the distal end 32 of the endoscope 14 as the endoscope 14 is withdrawn. For example, the fluid management system controller 46 may be configured to compare the measured pressure (at pressure sensor 74) to the second threshold pressure P_{f}, as shown at block 336. If the measured pressure is less than the second threshold pressure P_{f}, the fluid management system controller 46 may be configured to issue an alert to the user indicating they should slow or reduce the speed at which they are withdrawing the endoscope 14, as shown at block 338. The alert may be a plain language message displayed on any of the displays 24, 36, 48, 70 of the operating room environment 10, an audio alert, a haptic alert, a visual alert (blinking light, color change, etc.), or the like. After the user has been alerted to slow the withdrawal speed or if the measured pressure is equal to or greater than the second threshold pressure P_{f}, the fluid management system controller 46 be configured to compare the measured pressure (at pressure sensor 74) to the desired pressure Pₛ, as shown at block 340.

If the measured pressure (at pressure sensor 74) is greater than the desired pressure Pₛ, the fluid management system controller 46 may be configured to gradually turn on the inflow pump 50 and/or the outflow pump 56 to control the pressure at the pressure sensor 74 to be less than the desired pressure Pₛ and greater than the second threshold pressure P_{f}, as shown at block 342. It is contemplated that activating only the outflow pump 56 may cause the access sheath 98 to collapse upon itself hindering debris removal. If the measured pressure (at pressure sensor 74) is less than the desired pressure Pₛ or when the pumps 50, 56 have been turned on to reduce the measured pressure, the fluid management system controller 46 may then check the position of the endoscope 14 to determine if the endoscope 14 has been fully withdrawn from the access sheath 98, as shown at block 344. If the endoscope 14 has been fully withdrawn from the access sheath 98, the stones and/or stone fragments are deposited in the collection container 88, as shown at block 346, and the procedure is complete. If the endoscope 14 has not been fully withdrawn from the access sheath 98, the fluid management system controller 46 is configured to return to step 330 to determine if the position of the endoscope 14 and control the inflow pump 50 and outflow pump 56 based on the position of the endoscope 14 and the measured pressure until the endoscope 14 is fully withdrawn.

The computing systems 22, 68, and/or controllers 46 described herein may take many forms, including, for example, a microcontroller or microprocessor, coupled to a memory storing readable instructions for performing methods as described herein, as well as providing configuration of the computing system and/or controller for the various examples that follow. The computing system and/or controller may include one more application-specific integrated circuits (ASIC) to provide additional or specialized functionality, such as, without limitation a signal processing ASIC that can filter received signals from one or more sensors using digital filtering techniques. Logic circuitry, state machines, and discrete or integrated circuit components may be included as well. The skilled person will recognize many different hardware implementations are available for a computing system and/or controller.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoscope system, comprising:
an endoscope having a pressure sensor at a distal end;
an access sheath defining a lumen extending from a proximal end to a distal end thereof and configured to movably receive the endoscope within the lumen; and
a fluid management system, comprising:
an inflow pump fluidly coupled to the endoscope;
an outflow pump fluidly coupled to the access sheath; and
a controller configured to control the inflow pump and outflow pump based on pressure measurements from the pressure sensor and a position of the distal end of the endoscope relative to the distal end of the access sheath.

2. The endoscope system of claim 1, wherein the access sheath includes markings at fixed intervals along its length.

3. The endoscope system of claim 2, wherein the endoscope includes a camera configured oriented towards the markings.

4. The endoscope system of claim 3, wherein the controller is configured to identify a position of the distal end of the endoscope based on a count of the markings.

5. The endoscope system of claim 1, further comprising a linear encoder positioned adjacent to the proximal end of the access sheath.

6. The endoscope system of claim 5, wherein the endoscope includes markings at fixed intervals along its length.

7. The endoscope system of claim 1, wherein the controller is configured to maintain different pressure thresholds based on the position of the distal end of the endoscope.

8. The endoscope system of claim 7 wherein the controller is configured to maintain a first pressure threshold when the distal end of the endoscope extends distally beyond a distal end of the access sheath and a second pressure threshold when the distal end of the endoscope is proximal to the distal end of the access sheath.

9. The endoscope system of claim 8, wherein the second pressure threshold is less than the first pressure threshold.

10. The endoscope system of claim 8, wherein the controller is configured to calculate the second pressure threshold based at least in part on a fluid velocity within the access sheath.

11. The endoscope system of claim 8, wherein the controller is configured to maintain a third pressure threshold during withdrawal of large stone fragments, wherein the third pressure threshold is between the first and second pressure thresholds.

12. An endoscope system, comprising:
an endoscope having a pressure sensor at a distal end;
an access sheath defining a lumen extending from a proximal end to a distal end thereof and configured to movably receive the endoscope within the lumen;
a fluid management system comprising:
a controller;
an inflow pump configured to be fluidly coupled to the endoscope; and
an outflow pump fluidly coupled to the access sheath;
wherein the controller is configured to:
receive a maximum allowable pressure value;
measure an initial pressure value via the pressure sensor;
control the inflow pump and the outflow pump to maintain a measured pressure below the maximum allowable pressure value; and
adjust operation of the inflow pump and the outflow pump based on a position of the distal end of the endoscope relative to a distal end of the access sheath.

13. The endoscope system of claim 12, wherein the controller is configured to maintain the measured pressure at a first threshold when the distal end of the endoscope extends distally beyond the distal end of the access sheath and at a second threshold when the distal end of the endoscope is within the access sheath.

14. The endoscope system of claim 13, wherein the second threshold is less than the first threshold.

15. The endoscope system of claim 12, wherein the controller is configured to issue an alert when a withdrawal speed of the endoscope exceeds a threshold value.

16. A method of controlling fluid flow during a medical procedure, the method comprising:
receiving a maximum allowable pressure value;
measuring an initial pressure via a pressure sensor at a distal end of an endoscope;
controlling an inflow pump and an outflow pump to maintain a measured pressure at or below the maximum allowable pressure value;
determining a position of the distal end of the endoscope relative to a distal end of an access sheath; and
adjusting operation of the inflow pump and the outflow pump based on the determined position.

17. The method of claim 16, further comprising:
maintaining the measured pressure at a first threshold when the distal end of the endoscope extends beyond the distal end of the access sheath; and
maintaining the measured pressure at a second threshold when the distal end of the endoscope is within the access sheath.

18. The method of claim 16, wherein determining the position comprises detecting markings on the access sheath with a camera.

19. The method of claim 16, wherein determining the position comprises using a linear encoder.

20. The method of claim 16, further comprising detecting a direction of motion and speed of the endoscope relative to the access sheath.
